(19) United States
(12) Patent Application Publication
(10) Pub. No.: US 2024/0000481 A1
(43) Pub. Date: Jan. 4, 2024

(54) RETENTION HINGE FOR INSERTABLE PHYSIOLOGICAL MONITOR INJECTOR TOOL

(71) Applicant: Bardy Diagnostics, Inc., Bellevue, WA (US)

(72) Inventors: Mercer Peterson, Seattle, WA (US); Daniel L. Reddy, Seattle, WA (US); Jared Floyd, Ferndale, WA (US)

(21) Appl. No.: 18/341,997

(22) Filed: Jun. 27, 2023

Related U.S. Application Data
(60) Provisional application No. 63/357,445, filed on Jun. 30, 2022.

(51) Int. Cl. A61B 17/34; A61B 5/00

(52) U.S. Cl. CPC A61B 17/3468; A61B 5/686; A61B 2560/063; A61B 2017/347

(57) ABSTRACT

An insertable physiological monitor injector tool is provided. An elongated handle includes a recess formed along a longitudinal axis and has an opening on a distal end. An insertion tube has a hollow elongated shape that is movably positioned within the elongated handle, in the recess. A retention hinge is cut from a surface of the insertion tube and formed as a curve in an interior of the tube. A stationary arbor is affixed on a proximal end to a proximal end of the elongated handle and extends through the insertion tube when the insertion tube is in a retracted position. A tab is affixed to the insertion tube, wherein the tab can lock the insertion tube in an extended position.

### PRIORITY CLAIM AND CROSS-REFERENCE TO RELATED APPLICATIONS

This application claims priority to and the benefit of U.S. Provisional Patent Application No. 63/357,445, filed Jun. 30, 2023, titled RETENTION HINGE FOR INSERTABLE PHYSIOLOGICAL MONITOR INJECTOR TOOL, the entire contents of which are incorporated by reference herein in their entirety and relied upon.

### FIELD

This application relates in general to medical tools, and in particular to a retention hinge for an insertable physiological monitor injector tool.

### BACKGROUND

Medical diagnosis includes evaluating patient physiology, which describes the vital functions of the patient's anatomical structure, that is, the living body and its organs. A patient's physiology is determined through medical diagnostic procedures that include performing medical tests and, when available, reviewing patient data that has been collected through monitoring, although the data should first be correlated to patient symptoms to be of relevant diagnostic value.

Sporadic conditions present a special challenge because diagnostic tests performed in a physician's office may prove ineffective if the sporadic condition fails to present while the test is being performed. Sporadic conditions may be due to chronic or acute cause and can include transient signs, such as erratic heartbeat, muscle or nerve spasms, or hypoglycemia (or hyperglycemia) that may be accompanied by discernable symptoms. The unpredictable nature of sporadic conditions often makes the capturing of physiological data a matter of good timing. If the sporadic condition fails to occur during the course of a medical test, no physiological data, and therefore no diagnostic insight, is obtained.

In response, physicians have turned to ambulatory monitoring, which utilizes sensors placed cutaneously on or implanted within a patient's body that are attached to a recorder to provide physiological data capture while the patient goes about daily life.

Ambulatory monitors that are either wholly implanted inside the patient's body or which use implanted sensors will generally provide cleaner physiological data relatively free of environmental noise and effects, especially when compared to data captured cutaneously. However, a tool or other means for performing implantation of the ambulatory monitor is required. Implantation can be invasive to some degree and carries more risk than cutaneous or external forms of ambulatory monitoring. Generally, when inserting an implantable medical device into a patient, some sort of insertion tube is required to guide the device through an incision and into a subcutaneous pocket. Once the tube, with the device inside, is inside the pocket, the tube can be retracted to deposit the device inside. The tube must be easy to insert through the incision and there must be some type of retention feature at the end of the tube so that the device does not fall out of the insertion tube before being guided to the appropriate location for insertion.

Current implantation tools can include a distal retaining tab that extends from a sidewall of a cannula with one end attached to the sidewall and the opposite end of the tab free from and extending from the sidewall to releasably retain an object in the cannula. However, while the tab works to retain the object until the object is ready for insertion, the tab prevents loading of the object into the cannula from the same end at which the object is to be released.

Another implantation tool is described by U.S. Patent No. 2018/0168686, and includes a housing member with a pair of opposite extending recesses to house retaining elements. Each retaining element is coupled to the housing member.

As described above, both implantations tools can likely prevent an object from unintentional release.

Therefore, a need remains for an implant tool that safely and quickly injects a medical implant in a patient, while allowing the medical implant to be inserted and released at a certain location in a patient via a same end of the implant tool, while preventing unintentional release of the medical implant.

### SUMMARY

An insertable physiological monitor injector tool includes a handle within which an arbor and insertion tube are positioned. The insertion tube can have a hollow cylindrical form and be sized to house the arbor. A button can be positioned on an outer surface of the handle to retract the insertion tube when pressed or pulled back. During retraction of the insertion tube, the arbor remains stationary. The insertion tube is extended and preloaded with an insertable physiological monitor (IPM), such that one end of the IPM is adjacent to a distal end of the arbor. Examples of the IPM can include monitors for cardiac, oxygen, blood pressure, and glucose, as well as other types of monitors.

The insertion tube is inserted into the skin of a patient, at an angle, and the injector tool is rotated to lay flat along the skin to lift the skin up while performing a tunneling action. Once the insertion tube has reached a desired location for the IPM, the button is pressed to retract the insertion tube. The stationary arbor ensures the IPM remains in place, in the patient by preventing movement of the IPM with the insertion tube as the insertion tube retracts. Subsequently, injector tool is removed from the patient.

An embodiment provides an insertable physiological monitor injector tool. An elongated handle includes a recess formed along a longitudinal axis and has an opening on a distal end. An insertion tube has a hollow elongated shape that is movably positioned within the elongated handle, in the recess. A stationary arbor is affixed on a proximal end to a proximal end of the elongated handle and extends through the insertion tube when the insertion tube is in a retracted position. A tab is affixed to the insertion tube, wherein the tab can lock the insertion tube in an extended position.

A further embodiment provides an insertable physiological monitor injector tool having a structure through which a monitor housed in the structure is rechargeable.

In light of the disclosure set forth herein, and without limiting the disclosure in any way, in a first aspect of the present disclosure, which may be combined with any other aspect, or portion thereof, an insertable physiological monitor injector tool includes a structure through which a monitor housed in the structure is rechargeable, and a retention hinge cut from a surface of the structure and formed as a curve in an interior of the structure.

In a second aspect of the present disclosure, which may be combined with any other aspect, or portion thereof, the structure comprises an insertion tube having a hollow elongated shape.

In a third aspect of the present disclosure, which may be combined with any other aspect, or portion thereof, the tool further includes at least one of an elongated handle, a stationary arbor, and a tab. The elongated handle within which a recess is formed along a longitudinal axis and comprising an opening on a distal end. The structure is housed in the recess. The stationary arbor is affixed on a proximal end to a proximal end of the elongated handle and extends through the insertion tube when the insertion tube is in a retracted position. The tab is affixed to the insertion tube, wherein the tab can lock the insertion tube in an extended position.

In a fourth aspect of the present disclosure, which may be combined with any other aspect, or portion thereof, an insertable physiological monitor injector too includes an elongated handle, an insertion tube, a stationary arbor, and a tab. The elongated handle within which a recess is formed along a longitudinal axis and comprising an opening on a distal end. The insertion tube comprising a hollow elongated shape that is movably positioned within the elongated handle, in the recess. The stationary arbor affixed on a proximal end to a proximal end of the elongated handle and extends through the insertion tube when the insertion tube is in a retracted position. The tab affixed to the insertion tube, wherein the tab can lock the insertion tube in an extended position.

In a fifth aspect of the present disclosure, which may be combined with any other aspect, or portion thereof, further including an implantable physiological monitor placed within the insertion tube when the insertion tube is in the extended position.

In a sixth aspect of the present disclosure, which may be combined with any other aspect, or portion thereof, a channel is formed within a portion of the handle, on the proximal end, to allow movement of the tab.

In a seventh aspect of the present disclosure, which may be combined with any other aspect, or portion thereof, the insertion tube is configured to retract into the handle via the recess upon movement of the tab in the channel towards a proximal end of the handle.

In an eighth aspect of the present disclosure, which may be combined with any other aspect, or portion thereof, the insertion tube is made from plastic.

In a ninth aspect of the present disclosure, which may be combined with any other aspect, or portion thereof, a proximal end of the insertion tube is sharpened.

In a tenth aspect of the present disclosure, which may be combined with any other aspect, or portion thereof, further comprising one or more sets of guides formed on an interior surface of the insertion tube and shaped to surround at least a portion of the insertion tube in the recess.

In an eleventh aspect of the present disclosure, which may be combined with any other aspect, or portion thereof, further comprising a tip formed on a distal end of the insertion tube.

In a twelfth aspect of the present disclosure, which may be combined with any other aspect, or portion thereof, further comprising a notch formed on a proximal end of the insertion tube.

In a thirteenth aspect of the present disclosure, which may be combined with any other aspect, or portion thereof, the button is affixed to the notch of the insertion tube.

In a fourteenth aspect of the present disclosure, which may be combined with any other aspect, or portion thereof, an insertable physiological monitor injector tool system includes an elongated handle, an insertion tube, a retention hinge, a stationary arbor, a tab, an insertable physiological monitor, and a charging device. The elongated handle within which a recess is formed along a longitudinal axis. The insertion tube comprising a hollow elongated shape that is movably positioned within the elongated handle, in the recess. The retention hinge cut from a surface of the insertion tube and formed as a curve in an interior of the structure. The stationary arbor affixed on a proximal end to a proximal end of the elongated handle and extends through the insertion tube when the insertion tube is in a retracted position. The tab affixed to the insertion tube, wherein the tab can lock the insertion tube in an extended position. The insertable physiological monitor placed within the insertion tube when the insertion tube is in the extended position. The charging device to recharge a battery of the insertable physiological monitor when located in the insertion tube.

In a fifteenth aspect of the present disclosure, which may be combined with any other aspect, or portion thereof, further comprising an incision tool comprising a handle and a blade affixed to a distal end of the handle.

In a sixteenth aspect of the present disclosure, which may be combined with any other aspect, or portion thereof, further comprising a tunneller tool comprising a handle and a rod affixed to a distal end of the handle.

In a seventeenth aspect of the present disclosure, which may be combined with any other aspect, or portion thereof, the insertion tube is plastic.

In an eighteenth aspect of the present disclosure, which may be combined with any other aspect, or portion thereof, further comprising a tip formed on a distal end of the insertion tube.

In a nineteenth aspect of the present disclosure, which may be combined with any other aspect, or portion thereof, further comprising a notch formed on a proximal end of the insertion tube.

In a twentieth aspect of the present disclosure, which may be combined with any other aspect, or portion thereof, the button is affixed to the notch of the insertion tube.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, including time and clustering of events, all without departing from their spirit and the scope. Accordingly, the drawings and detailed description are to be regarded as illustrative in nature and not as restrictive.

### BRIEF DESCRIPTION OF THE DRAWINGS

FIG. 1 illustrates a perspective view of an insertable physiological monitor injector tool, according to an example embodiment of the present disclosure.

FIG. 2 illustrates an interior view of a cross section of a handle of the insertable physiological monitor injector tool of FIG. 1, according to an example embodiment of the present disclosure.

FIG. 3 illustrates a perspective view of an insertion tube of the insertable physiological monitor injector tool of FIG. 1, according to an example embodiment of the present disclosure.

FIG. 4 illustrates a perspective view of an incision tool, according to an example embodiment of the present disclosure.

FIG. 5 illustrates a perspective view of a tunneller tool, according to an example embodiment of the present disclosure.

FIG. 6 illustrates a perspective view of an insertable physiological monitor injector tool loaded with an insertable physiological monitor, according to an example embodiment of the present disclosure.

FIG. 7 illustrates a perspective view of an insertable physiological monitor injector tool in a partially retracted position, according to an example embodiment of the present disclosure.

FIG. 8 illustrates a perspective view of an insertable physiological monitor injector tool in a fully retracted position, according to an example embodiment of the present disclosure.

FIG. 9 illustrates a side view of an insertable physiological monitor injector tool in a fully retracted position, according to an example embodiment of the present disclosure.

FIG. 10 illustrates a top view of an insertable physiological monitor injector tool in a fully retracted position, according to an example embodiment of the present disclosure.

FIG. 11 illustrates a side view of an insertable physiological monitor injector tool in a fully retracted position, according to an example embodiment of the present disclosure.

FIG. 12 illustrates a top view of a different embodiment of the insertable physiological monitor injector tool of FIG. 1, according to an example embodiment of the present disclosure.

FIG. 13 illustrates a side view of a distal end of an insertion tube of the embodiment of the insertable physiological monitor injector of FIG. 12, according to an example embodiment of the present disclosure.

FIG. 14 illustrates a perspective view of the insertable physiological monitor injector tool of FIG. 8, according to an example embodiment of the present disclosure.

FIG. 15A illustrates a perspective view of an insertable physiological monitor injector tool with a channel, according to an example embodiment of the present disclosure.

FIG. 15B illustrates a perspective view of an insertable physiological monitor injector tool with a tab in a channel stem, according to an example embodiment of the present disclosure.

FIG. 15C illustrates a perspective view of an insertable physiological monitor injector tool with a tab on a distal end of a channel, according to an example embodiment of the present disclosure.

FIG. 15D illustrates a perspective view of an insertable physiological monitor injector tool with a tab on a proximal end of a channel, according to an example embodiment of the present disclosure.

FIG. 15E illustrates a side view of an insertable physiological monitor injector tool with a tab, according to an example embodiment of the present disclosure.

FIG. 16 illustrates a peripheral view of the insertion tube with a retention hinge, according to an example embodiment of the present disclosure.

FIG. 17 illustrates a side view of an interior of the insertion tube with the retention hinge, according to an example embodiment of the present disclosure.

FIG. 18 illustrates a side view of the retention hinge in the opening of the insertion tube, according to an example embodiment of the present disclosure.

FIG. 19 illustrates a top view of the insertion tube with the retention hinge in the opening, according to an example embodiment of the present disclosure.

### DETAILED DESCRIPTION

Implantable devices can provide patients with benefits, such as continuous monitoring of physiological signals, which can be helpful to detect sporadic events and facilitate diagnosis by a medical professional. However, injecting the implantable device can be invasive and come with some risk to the patient, such as infection or adverse reaction due to penetrating the patient's skin during injection. An injection tool that is minimally invasive, such as requiring a small incision, and is quick and efficient for implantation can help reduce risk to the patient.

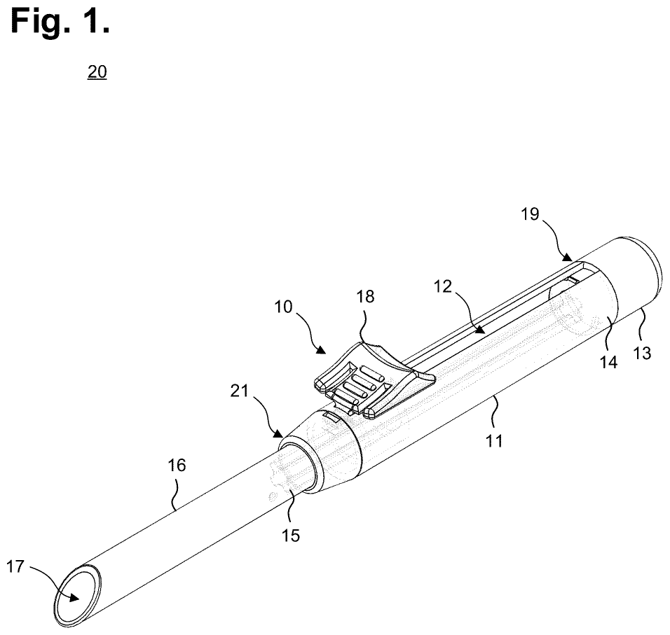

The minimally invasive injector tool can include an insertion tube, a handle, and an arbor. FIG. 1 illustrates, by way of example, a perspective view 10 of an insertable physiological monitor injector tool 10. The injector tool 10 can include a handle 11 with a recess formed in an interior of the handle, along a longitudinal axis. The handle 11 can have a cylindrical semi-circular or rectangular shape, as well as other types of shapes. In one embodiment, a shape of the handle can be based on a shape of an implantable physiological monitor (IPM) to be injected into a patient using the injection tool. A proximal end 19 of the handle can be enclosed, while a distal end 21 of the handle can be open to allow access to the recess.

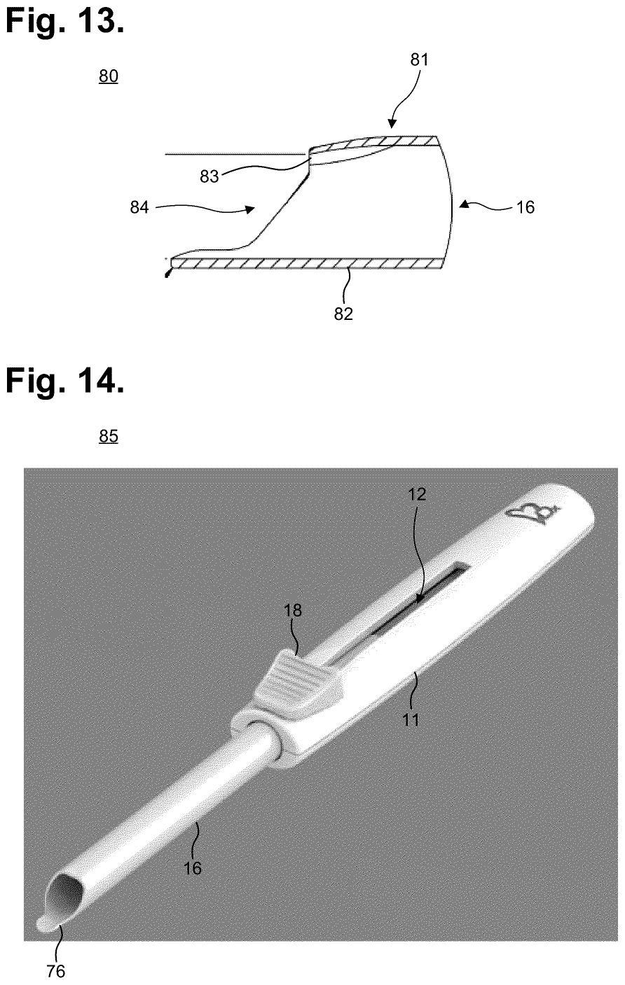

An insertion tube 16, which can have a different shape or the same shape as the handle, is provided in the recess of the handle. The insertion tube 16 can have a hollow interior 17 and be sized to fit in the recess 12 of the handle. At a minimum, a length of the insertion tube 16 should extend just beyond a distal end 21 of the handle via the opening. A distal end of the insertion tube can include a beveled edge or a straight edge, and can be sharpened or unsharpened. In a further embodiment, a tip can be attached to the distal end of the injector tube, as further described below with respect to the FIGS. 12 and 13.

A button or tab 18 is affixed to the insertion tube 16 to allow a user to move the insertion tube 16 within the recess. The button or tab 18 is positioned over an outer surface of the handle 11 via a channel 22 formed by a cutout in the handle 11. The button can slide back and forth within the channel 22 to move the insertion tube 16 back and forth within the recess of the handle 11.

An arbor 15 fits within the insertion tube 16 and is affixed on one end to a proximal end 19 of the handle 11 or to a separate end piece 13 attached to the proximal end of the handle. In one embodiment, a proximal end of the arbor 15 is affixed to a support mount 14 that is then affixed to the proximal end of the handle or the separate end piece 13. The arbor 15 can be shaped as a circle, square, rectangle, oval, cross, or a different shape, and sized to fit within the insertion tube 16. A length of the arbor 15 can be dependent on a length of the handle and should extend slightly past the distal end of the handle 11. The arbor 15 remains in a fixed position, while the insertion tube 16 slides back and forth over the arbor 15.

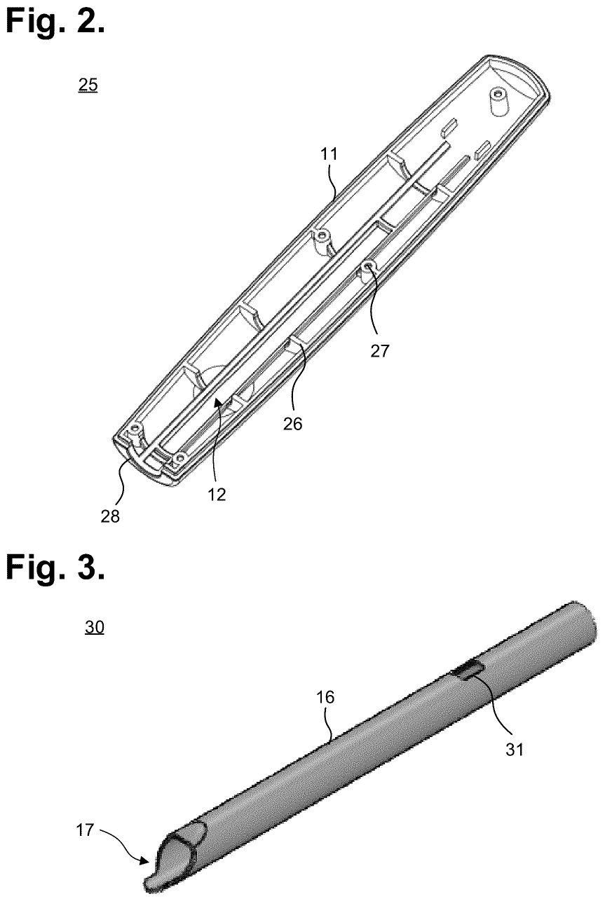

To ensure smooth movement of the insertion tube 16 within the handle 11, the handle can be conformed to fit securely around the insertion tube 16 with a slight space between an interior surface of the handle 11 and an exterior of the insertion tube 16. When the shape of the handle 11 differs from the insertion tube 16, an interior of the handle 11 can include guides to allow smooth movement of the insertion tube 16. FIG. 2 illustrates, by way of example, an interior view 25 of a cross section of a handle of the insertable physiological monitor injector tool of FIG. 1. The handle 11 can be formed from a single piece of material or from two or more pieces. The material can include plastic, metal, or stainless steel, as well as other types of material. In an interior of the handle 11, guides 26 can be formed along the recess 12 to guide the insertion tube (not shown) along the longitudinal axis of the recess 12. The guides can be formed as tabs of material, such as plastic, metal, or stainless steel, that extend from an interior surface of the handle 11. The guides can extend around or partially around the insertion tube (not shown). One or more sets of guides can be provided in the handle and each set can include two guides, which are each formed on either side of the insertion tube.

When the handle is formed from multiple pieces of material, screw holes or other fastening mechanism 27 can be formed along an interior of the handle. Screw holes or other fastening mechanism located on another piece of the handle should correspond with the screw holes on a first piece to secure the different pieces of the handle 11 together. At least one end of the handle is open 28, including the proximal end 21 of the handle to allow movement of the insertion tube (not shown) in and out of the handle 11.

The insertion tube slides within the recess via a button or tab. FIG. 3 illustrates, by way of example, a perspective view 30 of an insertion tube 16. The insertion tube 16 can be formed as a cylindrical tube with a hollow interior. However, other shapes are possible. Both ends of the insertion tube 16 can be open 17 to allow movement of the insertion tube 16 over the arbor (not shown). A cutout 31 is formed in a surface of the insertion tube 16 to affix to the button (not shown). For example, the button (not shown) can include a tab that is inserted into and secured in the cutout to allow a user to slide the button and thus, the insertion tube 16.

The insertion tube 16 can be made from material, including metal, stainless steel, plastic, or other types of material. When made from plastic, the IPM can be recharged while inserted in the insertable physiological monitor injector tool 10, as described below in further detail. A length of the insertion tube can be dependent on a location of an implantation site at which the IPM will be implanted. For example, an implantable cardiac monitor can be implanted between the skin and ribs of a wearer. Accordingly, the insertion tube 16 must be long enough to extend past the skin of a patient, but not longer than a distance between the ribs and the skin. Further, a shape of the insertion tube can be dependent on the IPM to be inserted in a wearer. For example, the insertion tube can be cylindrical when the IPM is cylindrical.

Implantation of the IPM can utilize an incision tool, tunneller tool, and the insertable physiological monitor injector tool. FIG. 4 illustrates, by way of example, a perspective view 35 of an incision tool 36. The incision tool 36 includes a handle 37 and a blade 38. The blade can be in the shape of a triangle, rectangle, or another shape. When shaped as a triangle, a base of the triangle can be affixed to a distal end of the handle 37 and an apex of the triangle facing outward from the handle 37. The incision tool 36 is used to make an incision in the skin of a patient after the incision area has been cleaned and sanitized.

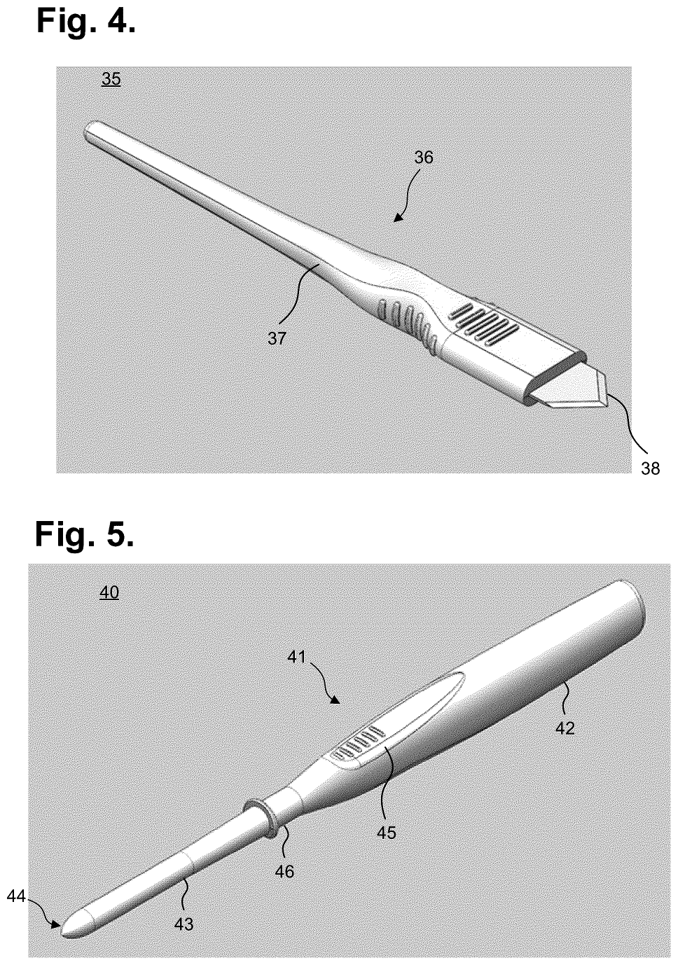

After an incision has been made, a tunneller tool is used to clear a path in the patient's tissue for later insertion of the insertable physiological monitor injector tool. FIG. 5 illustrates, by way of example, a perspective view 40 of a tunneller tool 41. The tunneller tool 41 includes a handle 42, a stopper or depth meter 46, and a rod 43. The handle 42 includes a grip 45 for a user when inserting the tunneller into a patient. The stopper or depth meter 46 is affixed on a distal end of the handle to provide a particular length of the rod which is to be inserted into the patient, while the rod 43 extends from the stopper. The stopper or depth meter 46 can be stationary or adjustable to adjust a length of the rod for different depth insertions in the patient. A distal end of the rod can include a tip 44 that can be sharpened or unsharpened. A length of the rod 43 can be dependent on a location of the IPM to be placed.

The tip 44 of the rod is inserted into the incision made by the incision tool. Once inserted, the user pushes the rod further into the patient via the handle 42 to clear a path in the tissue for placement of the IPM. The user can push the tunneller tool 41 into the patient until the stopper touches or is adjacent to the skin of the patient. Subsequently, the user removes the tunneller tool 41 from the patient.

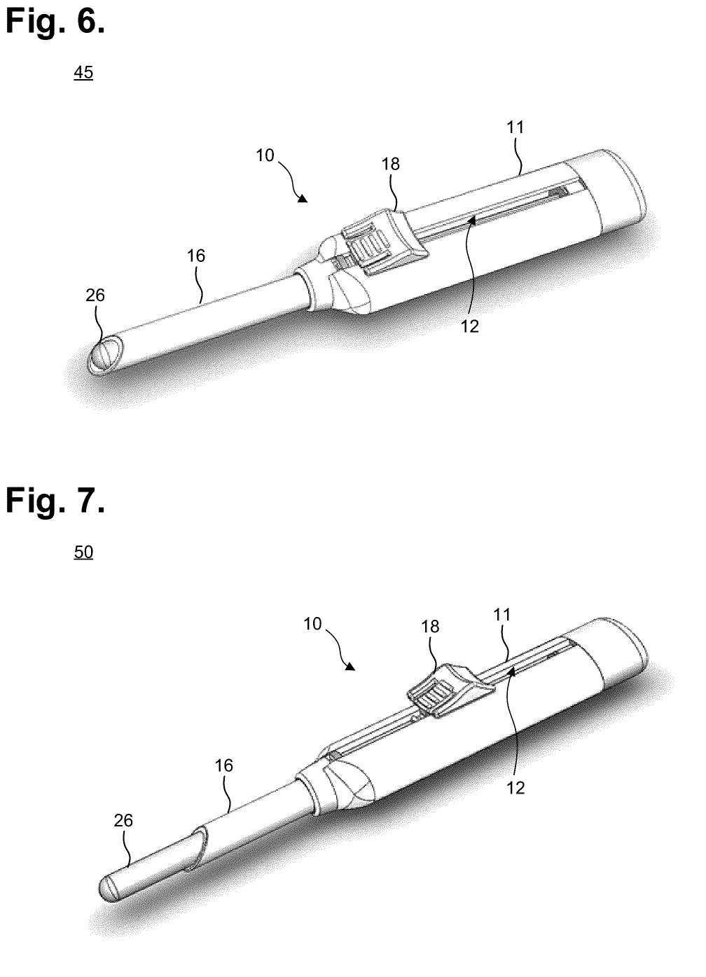

Once the incision has been made and a path is cleared for placement of the IPM, the insertable physiological monitor injector tool can be inserted into the patient. FIG. 6 illustrates, by way of example, a perspective view 45 of an insertable physiological monitor injector tool loaded with an IPM. For injection, the insertion tube 16 can be in an extended position, outside of the recess of the handle 11. An ICM 26 is placed within the insertion tube 16 and is adjacent on a proximal end to the arbor (not shown).

A distal end of the extended insertion tube 16 is inserted into the incision on the patient and a user moves the insertable physiological monitor injector tool into the tissue of the patient using the path formed by the tunneller tool. In one embodiment, the user can press down on or distally against the button 18 to prevent the insertion tube 16 from retracting towards a proximal end of the handle 11 via the recess 12. However, in a further embodiment, the button can be in a locked position to prevent retraction of the insertion tube 16.

Once a desired location has been reached by the distal end of the insertion tube 16, FIG. 7 illustrates, by way of example, a perspective view 50 of an insertable physiological monitor injector tool 10 in a partially retracted position. The insertable physiological monitor injector tool 10 can be inserted into the patient until the distal end of the housing touches or is adjunct to an outward facing surface of the patient's skin. Once reached, the button 18 can be pressed or released from a spring loaded or locked position, depending on a type of safety mechanism, and slowly moved along the channel 12, towards the proximal end of the handle 11. As the button is moved proximally, the insertion tube 16 slides over the IPM 26 which is stationary and begins to get exposed to the patient's tissue as the insertion tube retracts.

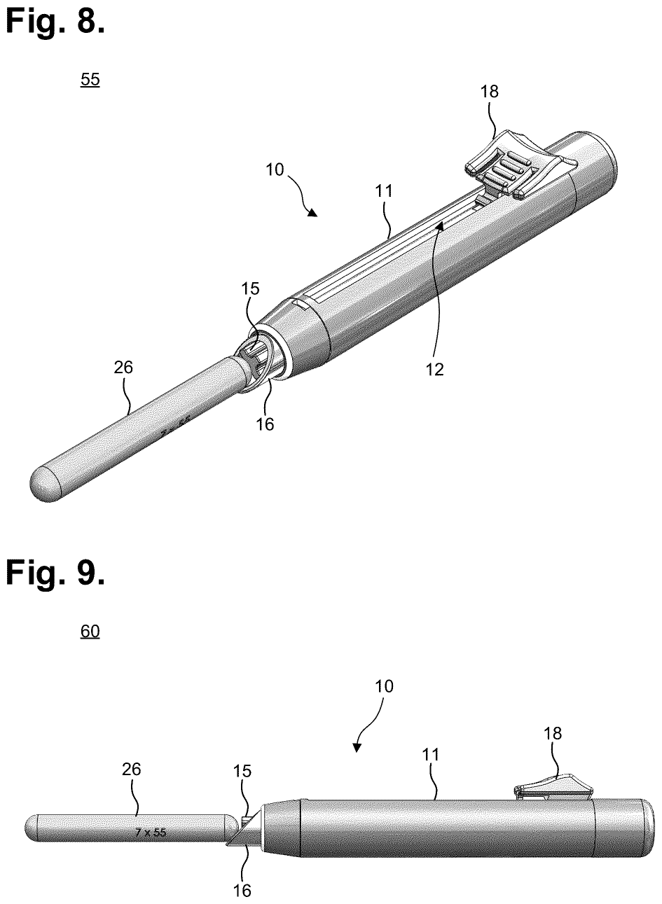

Once the insertion tube has been fully retracted, the IPM is positioned outside of the insertion tube and fully in the tissue of the patient. FIG. 8 illustrates, by way of example, a perspective view 55 of an insertable physiological monitor injector tool 10 in a fully retracted position. To obtain the retracted position, the user should slide the button 18 the length of the channel 22, formed in the handle 11, to move the insertion tube 16 along the recess 12 into an interior of the handle 11. As the insertion tube is retracted, more of the IPM 26 is revealed and introduced to the tissue of the patient. The stationary arbor 15 within the insertable tube is adjacent to a proximal end of the IPM 26 and prevents the IPM 26 from retracting with the insertable tube 16. Thus, the IPM 26 remains in the patient's tissue and the insertable physiological monitor injector tool 10 is moved away from the patient after the insertion tube 16 has been fully retracted.

FIG. 9 illustrates, by way of example, a side view 60 of an insertable physiological monitor injector 10 in a fully retracted position. In the fully retracted position, the button 18 is at the proximal end of the handle 11, the insertion tube 16 is fully retracted and a portion of the insertion tube 16 may extend slightly past the distal end of the handle. The arbor remains stationary and also extends slightly past the distal end of the handle, while the IPM 26 is no longer housed by the insertion tube 16 and is left in the tissue of the patient at the desired implantation site.

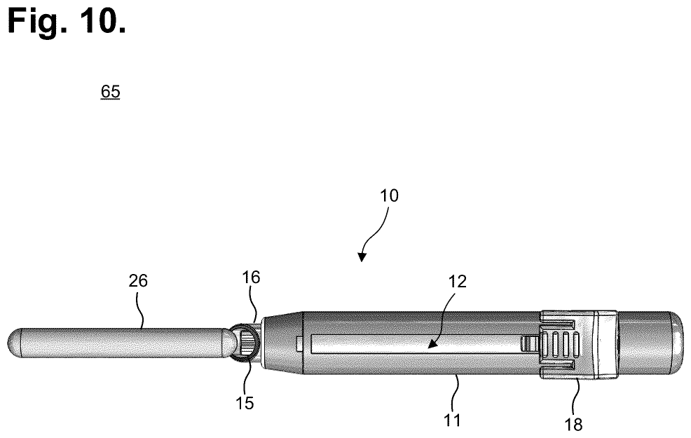

FIG. 10 illustrates, by way of example, a top view 65 of an insertable physiological monitor injector tool 10 in a fully retracted position. When the insertion tube 16 is fully retracted, the button 18 is on a proximal end of the handle and the IPM 26 is no longer in the insertable physiological monitor injector tool 10. The IPM 26 is positioned in the tissue of the patient and remains as the insertable physiological monitor injector tool is removed from the patient. The arbor 15 prevents the IPM 26 from retracting with the insertion tube 16 along the recess of the handle during retraction and removal of the insertable physiological monitor injector tool 10.

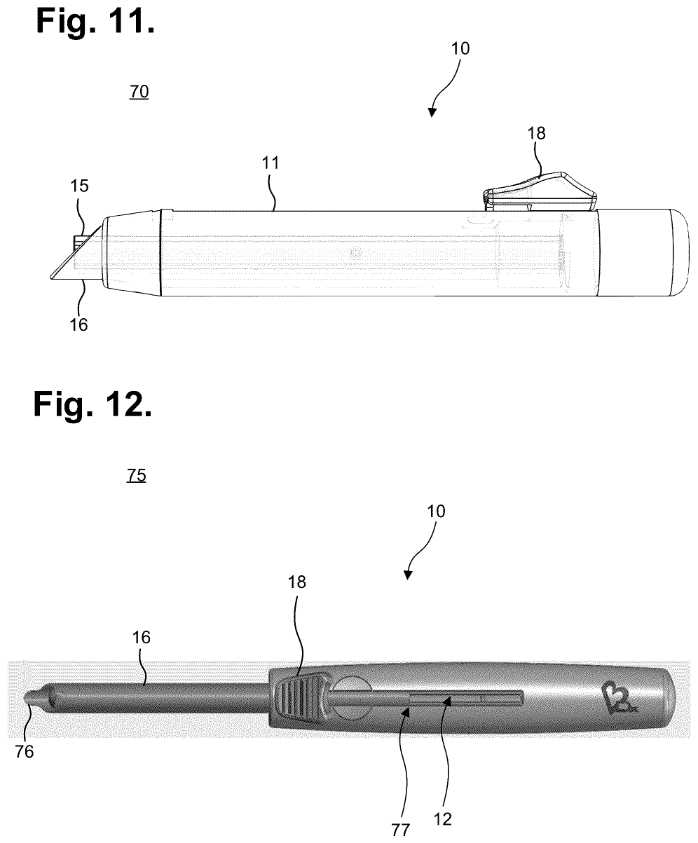

When in the fully retracted position, the arbor is positioned within the insertion tube and the insertion tube is within the recess of the handle. FIG. 11 illustrates, by way of example, a side view 70 of an insertable physiological monitor injector tool 10 in a fully retracted position. In the fully retracted position, the button 18 is on the proximal end of the insertable physiological monitor injector tool 10. The insertion tube 16 is positioned in the recess of the handle and the arbor 15 is positioned within the insertion tube 16.

The above description of the insertable physiological monitor injector tool can have different embodiments. For example, FIG. 12 illustrates, by way of example, a top view 75 of a different embodiment of the insertable physiological monitor injector 10 of FIG. 1. A tip 76 can be formed on at least a portion of the distal end of the insertion tube 16. The tip 76 can be heat formed to prevent the IPM (not shown) from falling out of the insertion tube 16 during insertion into the patient. FIG. 13 illustrates, by way of example, a side view 80 of a distal end of an insertion tubel 6 of the embodiment of the insertable physiological monitor injector of FIG. 12. A distal end 81 of the insertion tube 16 can include a tip 83 for preventing an IPM placed in an interior 84 of the insertion tube 16 from falling out, especially when the injector tool is angled for insertion into a patient.

In one embodiment, the tip 81 can be formed on an upper surface of the insertion tube, nearest to the button. The tip 83 can be formed inward, into the hollow interior 84, to produce interference between the insertion tube and the ICM. For example, the material 82 of the insertion tube 16 can bend downward on the distal tip, into the hollow interior 84. However, bending the tip 83 too far into an interior of the insertion tube can prevent release of the IPM when the insertion tube retracts. Accordingly, the tip 83 should be formed to prevent the IPM from falling out, but allow the insertion tube to retract over the IPM, which is stationary due to the arbor (not shown).

In a further embodiment, the tip can include an additional piece of material affixed to an upper interior surface of the insertion tube material 82. The additional material can extend downward into the hollow interior of the insertion tube to prevent the IPM from falling out.

When the insertion tube 16 is fully extended, the button 18 is on a distal end of the handle 11 and the recess 12 in the handle 11 can be visible. Also, a proximal end 77 of the insertion tube 16 can be visible through the channel. However, when in a fully retracted position, the recess 12 may not be visible since the outer surface of the insertion tube 16 can block a view of the recess 12 via the channel 22.

FIG. 14 illustrates, by way of example, a perspective view of the insertable physiological monitor injector of FIG. 12. In an extended position, the insertion tube 16 extends outward from the handle 11 and the button 18 is located on a distal end of the handle 11. The tip 76 extends from a distal end of the insertion tube 16 on a bottom surface of the opening. However, in a further embodiment, the tip can extend from a top surface or fully around the opening of the insertion tube 16 on the distal end. The tip can prevent the IPM (not shown) from moving outside of the insertion tube 16 when the insertable physiological monitor injector tool 10 is inserted into a patient.

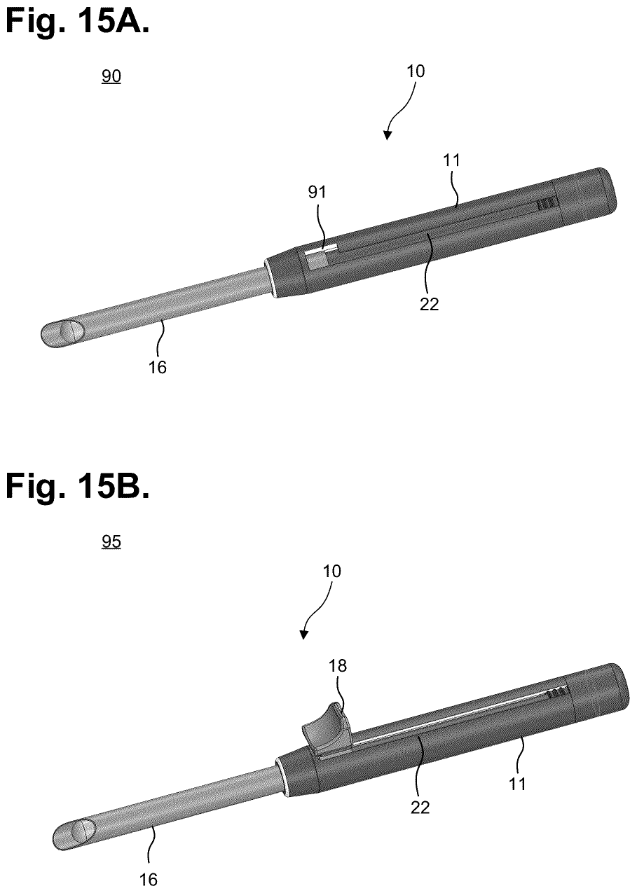

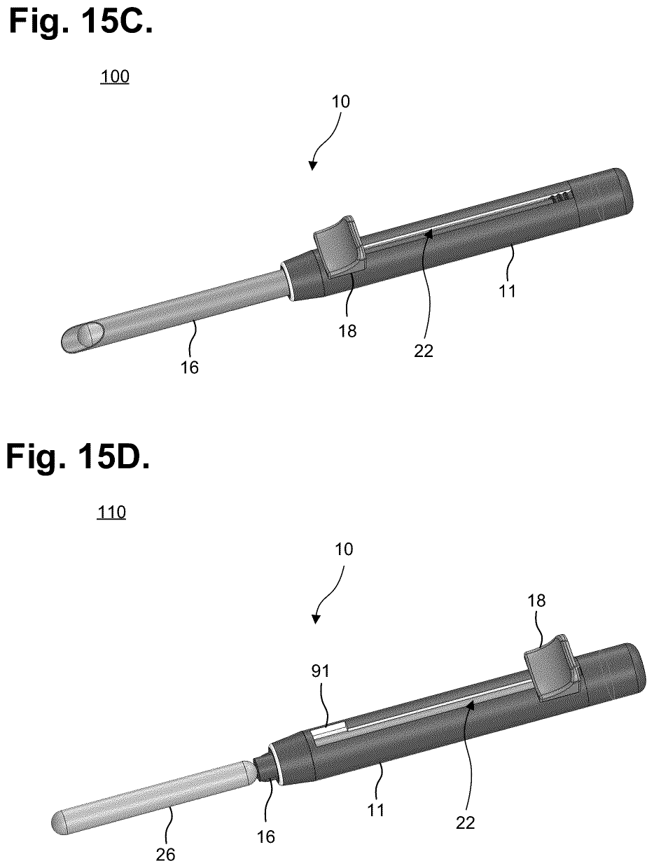

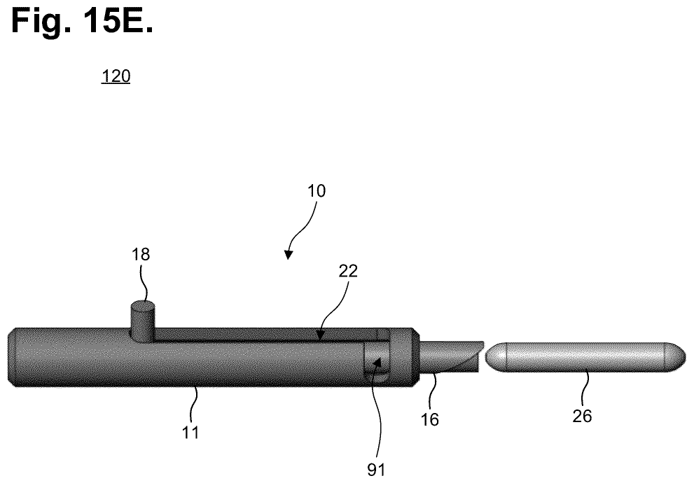

FIGS. 15A-E illustrate, by way of example, a different embodiment of the insertable physiological monitor injector of FIG. 1. FIG. 15A illustrates, by way of example, a perspective view 90 of an insertable physiological monitor injector tool 10 with a channel 22. The housing 11 of the insertable physiological monitor injector tool 10 has a channel 22 formed within a surface, such as a top surface. The channel 22 can run along a longitudinal axis of the housing 11 and include a stem 91, which is formed on a distal end of the housing and extends outwards, away from the channel along the longitudinal axis. The stem 91 can function to lock the insertion tube 16 in an extended position via a tab (not shown).

FIG. 15B illustrates, by way of example, a perspective view 95 of an insertable physiological monitor injector tool 10 with a tab 18 in a channel stem. A tab, such as a button 18 can be in a locked position when located in the stem (not shown) of the channel 22 formed in the handle 11. Specifically, when in the stem, the button 18 is prevented from moving distally or proximally along the handle, which prevents movement of the insertion tube 16. Thus, when the button or tab 18 is locked, the insertion tube 16 is locked in an extended position.

FIG. 15C illustrates, by way of example, a perspective view 100 of an insertable physiological monitor injector tool 10 with a tab 18 on a distal end of a channel 22. To unlock the insertion tube 16, of the insertable physiological monitor injector tool 10, from an extended position, a user slides a tab, such as a button 18 out of the stem (not shown) and into the channel 22. Specifically, a user moves the button or tab 18 across the longitudinal axis of the handle 11 and towards the channel 22.

FIG. 15D illustrates, by way of example, a perspective view 110 of an insertable physiological monitor injector tool 10 with a tab 18 on a proximal end of a channel 22. To retract the insertion tube 16, a user slides the tab, such as a button 18, away from the channel stem 91, towards a proximal end of the handle 11, along the channel 22. When the button 18 is on the proximal end, the insertion tube 16 moves into the recess of the handle and totally clears or mostly clears the IPM 26, leaving the IPM implanted in the tissue of a patient.

FIG. 15E illustrates, by way of example, a side view 120 of an insertable physiological monitor injector tool 10 with a tab 18. The tab can include a button, knob, pin, or other type of device that is capable of attachment to the insertion tube 16 and movement by the user. At a minimum the tab should be movable from a locked position in a channel stem 91, into a channel 22, and then along the channel 22. As the tab 18 is moved proximally along the channel 22, the insertion tube retracts into an interior of the housing 11, leaving the IPM 26 in the tissue of a patient.

In one embodiment, the IPM can be packaged with the insertable physiological monitor injector tool, such as by placing the IPM in the insertion tube. The injection tube is in the extended position to provide room in which the IPM can be placed on a distal end of the arbor, a portion of which is also inside the insertion tube in the extended position. Based on how much time passes from packaging of the IPM with the injector tool to injection of the IPM in a patient, battery power of the IPM may run low. Ideally, the IPM should be fully charged prior to insertion into the patient to ensure that all functions of the IPM are working. When the insertion tube is made from a conductive material that allows electromagnetic waves or energy to pass, the battery of the IPM can be recharged while remaining in the insertable physiological monitor injector tool, prior to placement in the patient. The material of the insertion tube can include plastic, metal or other types of material.

In one embodiment, the battery can be inductively charged via a wireless device that is placed over sterile packaging of the insertable physiological monitor injector tool. The wireless charging device can include a puck or wand or other type of device capable of wireless charging. The device wirelessly charges the battery until the battery is full. Subsequently, the insertable physiological monitor injector tool is removed from the package and used to insert the IPM in a patient.

The insertable physiological monitor injector tool can be constructed by building an elongated handle with a recess formed along a longitudinal axis in an interior of the handle. An insertion tube having a hollow elongated shape can be positioned within the recess of the elongated handle. One or more sets of guides are formed on an interior surface of the handle to surround and guide at least a portion of the insertion tube in the recess. In one embodiment, a notch can be formed on a proximal end of the insertion tube, which is moveable within the recess. A stationary arbor is placed within the insertion tube and affixed to a proximal end of the elongated handle, in the interior. When the insertion tube is in a retracted position, the stationary arbor extends through the insertion tube. A tab, such as a button or knob is affixed to the insertion tube via the notch. The tab can be used to lock the insertion tube in an extended position. An implantable physiological monitor is placed within the insertion tube when the insertion tube is in the extended position. A channel is formed within a portion of the handle, on the proximal end, to allow movement of the button to retract the insertion tube from the extended position to a retracted position.

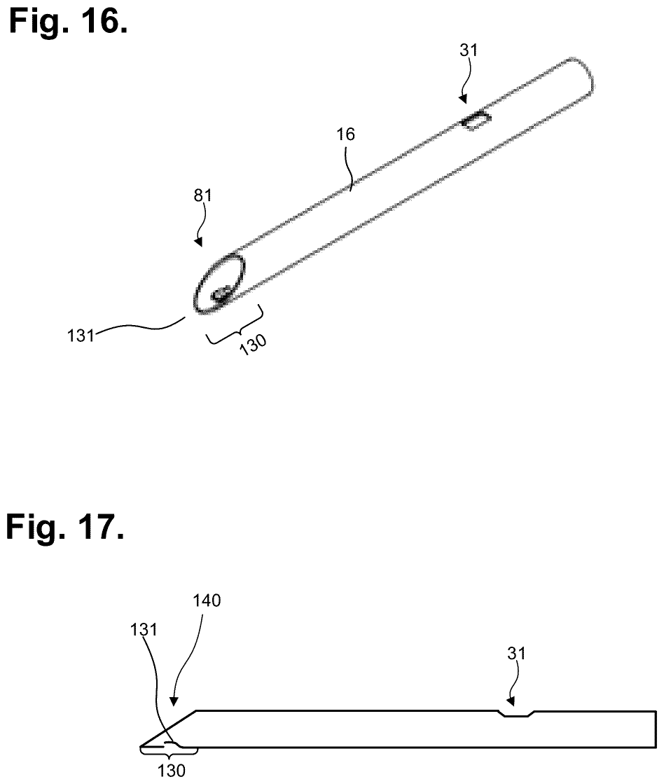

The IPM can be inserted into the insertion tube 16 from a front or back end. However, when inserted in the front or distal end, ensuring retention of the IPM in the insertion tube can become more difficult. A retention hinge or tongue can allow movement of the IPM into and out of the insertion tube, while ensuring that the IPM is not unintentionally released. FIG. 16 illustrates a peripheral view of the insertion tube 16 with a retention hinge 131. The insertion tube 16 can be cut at an angle on the distal end and the retention hinge or tongue 131 can be located on a longer exposed surface 130 of the insertion tube, which is formed by the cut. In one embodiment, the angle of the cut can be around 35 degrees from the axis of the tube; however, other angles are possible such as 25-45 degrees. The cut can be perpendicular to the axis of the tube.

The insertion tube 16 can be formed from a polyether ether ketone tube, although other materials are possible, such as other plastics or some metals, and can also be covered or coated in a friction-reducing material. A U-shape cut can be made in a surface of the insertion tube and curved to form the retention hinge 131, which is similar to a tongue in a mouth. For example, the rounded part of the U-shape is cut free from the insertion tube 16 and attached to the insertion tube on the attached end, which is opposite the rounded end. Other shapes of the retention hinge cut are possible including a rectangle.

The curve of the U-shape hinge can be formed in the interior of the insertion tube to prevent the IPM from unintentionally releasing from the tube, while also allowing the IPM to be inserted into the tube by a medical professional. In one embodiment, the retention hinge 131 is formed as a living hinge to contain the implantable monitor. The shape of the retention hinge 131 can resemble a hill or bell curve, where the attached end of the U-shape is formed upward into the interior of the insertion tube 16 and then bent downward, around a middle of the U-shape, so the free end of the U-shape ends slightly above the interior surface of the insertion tube 16 or is level with the interior surface of the insertion tube 16. Since the rounded end is free, the retention hinge 131 can move downwards to create more space in the interior of the insertion tube to allow the IPM in or out when pressure is applied to one of the sides of the curve. Once the pressure is removed, the retention hinge 131 moves back into place in the interior of the tube.

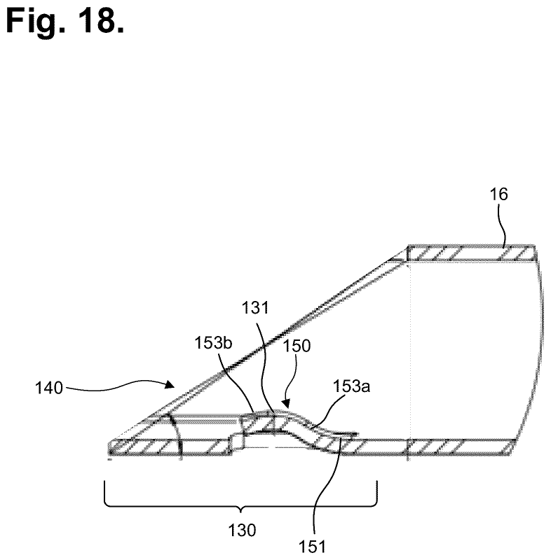

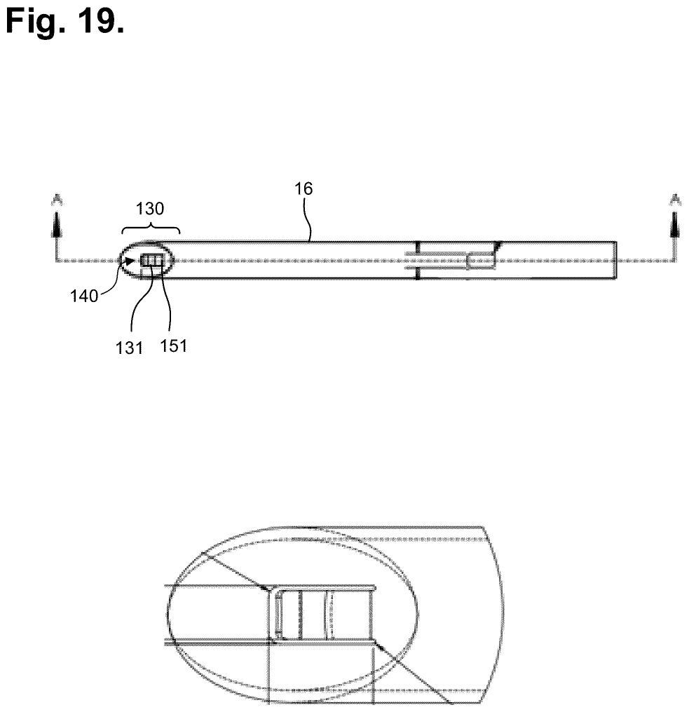

The retention hinge 131 can be shaped similar to a bell curve with a rounded top. FIG. 17 illustrates, by way of example, a side view of an interior of the insertion tube 16 with the retention hinge 131. The insertion tube 16 forms a hollow channel in which the IPM is placed for insertion in a patient. The distal end of the insertion tube 16 is cut at an angle perpendicular to an axis along the length of the insertion tube. The retention hinge 131 is formed in an opening 140 of the insertion tube, which is formed by the cut, and can have the shape of a curve. FIG. 18 illustrates, by way of example, a side view of the retention hinge in the opening of the insertion tube. The retention hinge 131 is formed on a surface of the insertion tube 16 that extends beyond 130 an opposite surface of the insertion tube 16, near the opening. Specifically, a U-shape or other shape is cut into the extended surface 130 of the insertion tube. FIG. 19 illustrates a top view of the insertion tube 16 with the retention hinge 131 in the opening 140. The retention hinge 131 is formed on the extended surface 130 of the insertion tube, along an axis A of the length of the insertion tube 16 and in line with the cutout 31. In one embodiment, the U-shape can have length of around 0.220 to 0.230 mm and a width around 0.120 to 0.130 mm. However, other lengths and widths are possible, especially depending on a size of the insertion tube. For example, a longer or wider insertion tube can have a longer, wider, or larger retention hinge.

One end of the hinge 131 is rounded and cut free from the insertion tube, while the other end is still attached to the insertion tube at a joint 151 located on a proximal end of the hinge 131, which is further from the opening than the rounded end on a distal end. The joint 151 allows the retention hinge 131 to move with respect to the insertion tube 16.

Returning to the discussion with respect to FIG. 18, the retention hinge 131 can be bent from the joint 151 into the insertion tube 16 to form a curve by sloping 153a to a crest 150 in the interior of the insertion tube, starting from the joint 151. The hinge then slopes 153b downward from the crest 150 and ends at the rounded end of the U-shape. The rounded end of the U-shape is free from the insertion tube and can move independent of the insertion tube to allow an IPM (not shown) to be inserted into or removed from the insertion tube 16. The free rounded end can extend above an interior surface of the insertion tube and may not touch the interior to allow movement. The curve size of the hinge can vary depending on the size of the insertion tube and IPM. However, at a minimum, the free rounded end of the U-shape hinge should not end too high above the interior surface of the insertion tube to prevent entrance of the IPM into the tube.

The IPM can be inserted into the insertion tube via the opening 140 by pressing the IPM against the slope 153b on a distal end of the hinge, closest to the opening. The pressure from a proximal end of the IPM, which is deepest in the insertion tube, presses the free end of the slope downward and away from the interior of the insertion tube. As the IPM moves further into the insertion tube, the IPM passes over the crest 150 and moves fully into the insertion tube. In one embodiment, a proximal end of the IPM touches the arbor, as described above with respect to FIG. 1. In a further embodiment, the IPM may not contact the arbor until the IPM is ready for release and the insertion tube 16 starts to retract.

One the distal end of the insertion tube passes the crest 150, the insertion tube is fully loaded and can be retained from falling out or unintentional release since the distal end cannot pass the crest 150 without pressure applied to the IPM. To remove the IPM, the insertion tube is inserted into the patient and once at a desired location, the insertion tube begins to retract. During retraction of the insertion tube, pressure is applied to a proximal end of the IPM by the arbor, which can cause a distal end of the IPM to push over the proximal slope 153a of the hinge and the crest 151 until the proximal end of the IPM is over the crest and released into the patient.

While the invention has been particularly shown and described as referenced to the embodiments thereof, those skilled in the art will understand that the foregoing and other changes in form and detail may be made therein without departing from the spirit and scope.

What is claimed is:

1. An insertable physiological monitor injector tool, comprising:
a structure through which a monitor housed in the structure is rechargeable; and
a retention hinge cut from a surface of the structure and formed as a curve in an interior of the structure.

2. The insertable physiological monitor injector tool according to claim 1, wherein the structure comprises an insertion tube having a hollow elongated shape.

3. The insertable physiological monitor injector tool according to claim 1, further comprising at least one of:
an elongated handle within which a recess is formed along a longitudinal axis and comprising an opening on a distal end, wherein the structure is housed in the recess;
a stationary arbor affixed on a proximal end to a proximal end of the elongated handle and extends through the insertion tube when the insertion tube is in a retracted position; and
a tab affixed to the insertion tube, wherein the tab can lock the insertion tube in an extended position.

4. An insertable physiological monitor injector tool, comprising:
an elongated handle within which a recess is formed along a longitudinal axis and comprising an opening on a distal end;
an insertion tube comprising a hollow elongated shape that is movably positioned within the elongated handle, in the recess;
a stationary arbor affixed on a proximal end to a proximal end of the elongated handle and extends through the insertion tube when the insertion tube is in a retracted position; and
a tab affixed to the insertion tube, wherein the tab can lock the insertion tube in an extended position.

5. The insertable physiological monitor injector tool according to claim 4, further comprising:
an implantable physiological monitor placed within the insertion tube when the insertion tube is in the extended position.

6. The insertable physiological monitor injector tool according to claim 4, wherein a channel is formed within a portion of the handle, on the proximal end, to allow movement of the tab.

7. The insertable physiological monitor injector tool according to claim 6, wherein the insertion tube is configured to retract into the handle via the recess upon movement of the tab in the channel towards a proximal end of the handle.

8. The insertable physiological monitor injector tool according to claim 4, wherein the insertion tube is made from plastic.

9. The insertable physiological monitor injector tool according to claim 4, wherein a proximal end of the insertion tube is sharpened.

10. The insertable physiological monitor injector tool according to claim 4, further comprising:
one or more sets of guides formed on an interior surface of the insertion tube and shaped to surround at least a portion of the insertion tube in the recess.

11. The insertable physiological monitor injector tool according to claim 4, further comprising:
a tip formed on a distal end of the insertion tube.

12. The insertable physiological monitor injector tool according to claim 4, further comprising:
a notch formed on a proximal end of the insertion tube.

13. The insertable physiological monitor injector tool according to claim 12, wherein the button is affixed to the notch of the insertion tube.

14. An insertable physiological monitor injector tool system, comprising:
an elongated handle within which a recess is formed along a longitudinal axis;
an insertion tube comprising a hollow elongated shape that is movably positioned within the elongated handle, in the recess;
a retention hinge cut from a surface of the insertion tube and formed as a curve in an interior of the structure;
a stationary arbor affixed on a proximal end to a proximal end of the elongated handle and extends through the insertion tube when the insertion tube is in a retracted position;
a tab affixed to the insertion tube, wherein the tab can lock the insertion tube in an extended position;
an insertable physiological monitor placed within the insertion tube when the insertion tube is in the extended position; and
a charging device to recharge a battery of the insertable physiological monitor when located in the insertion tube.

15. The system according to claim 14, further comprising:
an incision tool comprising a handle and a blade affixed to a distal end of the handle.

16. The system according to claim 14, further comprising:
a tunneller tool comprising a handle and a rod affixed to a distal end of the handle.

17. The system according to claim 14, wherein the insertion tube is plastic.

18. The system according to claim 14, further comprising:
a tip formed on a distal end of the insertion tube.

19. The insertable physiological monitor injector tool according to claim 14, further comprising:
a notch formed on a proximal end of the insertion tube.

20. The insertable physiological monitor injector tool according to claim 19, wherein the button is affixed to the notch of the insertion tube.
